(19) United States
(12) Patent Application Publication
(10) Pub. No.: US 2009/0099451 A1
(43) Pub. Date: Apr. 16, 2009

(54) ULTRASONIC IMAGING APPARATUS AND A METHOD FOR GENERATING AN ULTRASONIC IMAGE

(75) Inventors: Shigemitsu NAKAYA, Otawara-shi (JP); Akihiro KAKEE, Nasushiobara-shi (JP); Chihiro SHIBATA, Nasushiobara-shi (JP)

Correspondence Address: OBLON, SPIVAK, MCCLELLAND MAIER & NEUSTADT, P.C., 1940 DUKE STREET, ALEXANDRIA, VA, 22314, US

(73) Assignees: KABUSHIKI KAISHA TOSHIBA, Tokyo (JP); TOSHIBA MEDICAL SYSTEMS CORPORATION, Otawara-shi (JP)

(21) Appl. No.: 12/249,309

(22) Filed: Oct. 10, 2008

(30) Foreign Application Priority Data
Oct. 10, 2007 (JP) 2007-264318

(51) Int. Cl. A61B 8/14

(52) U.S. Cl. 600/443

(57) ABSTRACT

A transmitter transmits ultrasonic waves to an object via an ultrasonic probe. A receiver receives echo signals reflected from the object via the ultrasonic probe. The receiver executes a delaying process on the echo signals in accordance with a plurality of set sound velocities for a delaying process, thereby generating a plurality of reception signals with different set sound velocities. An image generator generates a plurality of image data with the different set sound velocities based on the reception signals with the different set sound velocities. A contrast calculator obtains the contrast of each of the plurality of image data with the different set sound velocities. A selector selects image data with the highest contrast from among the plurality of image data. A display controller controls a display to display an image based on the image data selected by the selector.

### BACKGROUND OF THE INVENTION

1. Field of the Invention

The present invention relates to an ultrasonic imaging apparatus that scans an object with ultrasonic waves and generates an ultrasonic image based on acquired reception signals, and also relates to a method for generating an ultrasonic image.

2. Description of the Related Art

An ultrasonic imaging apparatus employs a method of focusing a transmission beam and a reception beam in order to enhance the lateral resolution of an ultrasonic image. In particular, an electronic scanning ultrasonic imaging apparatus employs an electronic focusing method by a delaying process on transmission signals and reception signals in each channel.

The electronic focusing method has a problem such that a beam diffuses at a position (a depth) distant from a focusing point and thereby the lateral resolution decreases. Therefore, a dynamic focusing method is employed. The dynamic focusing method is a method of executing the delaying process so that the focusing point continuously shifts in the depth direction with time at the time of reception of ultrasonic waves. By this method, it is possible to acquire a reception beam from a region where a beam is focused.

Here, a delay time will be explained with reference to FIG. 1.

FIG. 1 is a schematic view for explaining a delay time Δt for focusing an ultrasonic beam. For example, assuming the coordinate in the depth direction of a focal point P is X and the coordinate in the lateral direction of an element within the reception aperture is Y, the origin of the coordinates is the center of the aperture, a time from the reach of the wave front of a reflected sound wave generated at the focal point P at the depth X for the center of the aperture to the reach thereof for the abovementioned element is the delay time Δt, and the sound velocity in the medium is C, the delay time Δt is expressed by the following formula (1).

Δt=((X²+Y²)^{1/2}−X)/C (1)

In an ultrasonic imaging apparatus according to the related art, the sound velocity C is set on the assumption of the representative sound velocity within a diagnosis site for imaging. Hereinafter, a sound velocity set in an ultrasonic imaging apparatus will be referred to as a “set sound velocity.” Then, a delay time is determined in accordance with the set sound velocity, and the delaying process is executed in accordance with the delay time. However, the value of a sound velocity in a living body (hereinafter referred to as a “living-body sound velocity”) varies depending on locations in the living body. For example, it is reported that the values of sound velocities are 1,560 cm/s in muscle and 1,480 cm/s in fat. Moreover, the living-body sound velocity differs among objects. Since the difference between the living-body sound velocity and the set sound velocity causes mismatch of the focusing points, the quality of ultrasonic images deteriorates.

For example, when the living-body sound velocity and the set sound velocity are equal, the delay time between ultrasonic transducers is set correctly, and therefore, the focusing points match. As a result, an ultrasonic image with high quality can be acquired. On the other hand, when the living-body sound velocity is higher than the set sound velocity, the delay time between the ultrasonic transducers is set large, and therefore, the focusing point becomes shallow. As a result, the lateral resolution of an ultrasonic image lowers. On the contrary, when the living-body sound velocity is lower than the set sound velocity, the delay time between the ultrasonic transducers is set small, and therefore, the focusing point becomes deep. As a result, the lateral resolution of an ultrasonic image lowers.

A technique for equalizing the set sound velocity and the living-body sound velocity has been proposed conventionally.

For example, there is a technique of executing scan for checking the set sound velocity before imaging for diagnosis and, based on the result of the scan, determining the value of the set sound velocity (e.g., Japanese Unexamined Patent Application Publication No. 2007-7045).

Then, by controlling the delay time in accordance with the set sound velocity, reception beams are generated.

Further, a plurality of ultrasonic images generated by controlling delay times using different set sound velocities are displayed simultaneously (e.g., Japanese Unexamined Patent Application Publication No. 2003-10180). In other words, a plurality of ultrasonic images obtained in accordance with different set sound velocities are displayed simultaneously.

However, the related art described in Japanese Unexamined Patent Application Publication No. 2007-7045 requires additional scan for checking the set sound velocity before imaging for diagnosis.

Therefore, a difference in time is caused between the check of the set sound velocity and the actual diagnosis. Thus, it is impossible to check and set the set sound velocity in real time at the time of the actual diagnosis. Moreover, since it is necessary to scan for checking the set sound velocity, there is a problem such that the duration for diagnosis gets long. Besides, in a case where a location for imaging is displaced at the time of imaging for diagnosis, it is necessary to execute scan for checking the set sound velocity again. Consequently, the duration for diagnosis gets long, and moreover, the check of the set sound velocity is required every time the imaging location is displaced, so that the operation is complicated.

The related art described in Japanese Unexamined Patent Application Publication No. 2003-10180 is merely the one that a plurality of ultrasonic images obtained in accordance with different set sound velocities are simultaneously displayed. Therefore, the operator needs to observe the plurality of ultrasonic images and select an image appropriate for diagnosis from among the plurality of ultrasonic images.

### SUMMARY OF THE INVENTION

An object of the present invention is to provide an ultrasonic imaging apparatus that can generate and display a high-resolution ultrasonic image without executing scan for checking the set sound velocity. Another object of the present invention is to provide a method by which a high-resolution ultrasonic image can be generated.

A first aspect of the present invention provides an ultrasonic imaging apparatus, comprising: a transmitter configured to transmit ultrasonic waves to an object via an ultrasonic probe; a receiver configured to receive echo signals reflected from the object via the ultrasonic probe, and execute a delaying process on the echo signals in accordance with a plurality of set sound velocities for the delaying process, thereby generating a plurality of reception signals with different set sound velocities; an image generator configured to generate a plurality of image data with the different set sound velocities, based on the reception signals with the different set sound velocities; a contrast calculator configured to obtain a contrast of each of the plurality of image data with the different set sound velocities; a selector configured to select image data with a highest contrast from among the plurality of image data; and a display controller configured to control a display to display an image based on the image data selected by the selector.

According to the first aspect, by execution of a delaying process in accordance with a plurality of set sound velocities, image data with different set sound velocities are generated, and the contrast of each of the image data is obtained. Then, an image based on image data with the highest contrast is displayed. Consequently, it is possible to generate and display a high-resolution image without executing scan for checking a set sound velocity.

Further, a second aspect of the present invention provides a method for generating an ultrasonic image, comprising: transmitting ultrasonic waves to an object via an ultrasonic probe; receiving echo signals reflected from the object via the ultrasonic probe; executing a delaying process on the echo signals in accordance with a plurality of set sound velocities for the delaying process, thereby generating a plurality of reception signals with different set sound velocities; generating a plurality of image data with the different set sound velocities, based on the reception signals with the different set sound velocities; obtaining a contrast of each of the plurality of image data with the different set sound velocities; selecting image data with a highest contrast from among the plurality of image data; and displaying an image based on the selected image data.

### BRIEF DESCRIPTION OF THE DRAWINGS

FIG. 1 is a schematic view for explaining a delay time Δt for focusing an ultrasonic beam.

FIG. 2 is a block diagram showing an ultrasonic imaging apparatus according to an embodiment of the present invention.

FIG. 3 is a block diagram showing a receiver installed in the ultrasonic imaging apparatus according to the embodiment of the present invention.

FIG. 4A is a view schematically showing tomographic images generated in accordance with different set sound velocities.

FIG. 4B is a view schematically showing tomographic images generated in accordance with different set sound velocities.

FIG. 4C is a view schematically showing a tomographic image.

FIG. 5 is a flow chart showing a series of operations by the ultrasonic imaging apparatus according to the embodiment of the present invention.

FIG. 6 is a view schematically showing tomographic images generated in accordance with different set sound velocities.

FIG. 7 is a flow chart showing a series of operations by an ultrasonic imaging apparatus according to Modification 1.

FIG. 8A is a view schematically showing an imaging region.

FIG. 8B is a view schematically showing tomographic images in the imaging region.

FIG. 9 is a flow chart showing a series of operations by an ultrasonic imaging apparatus according to Modification 2.

FIG. 10 is a view schematically showing tomographic images in individual regions adjacent to each other.

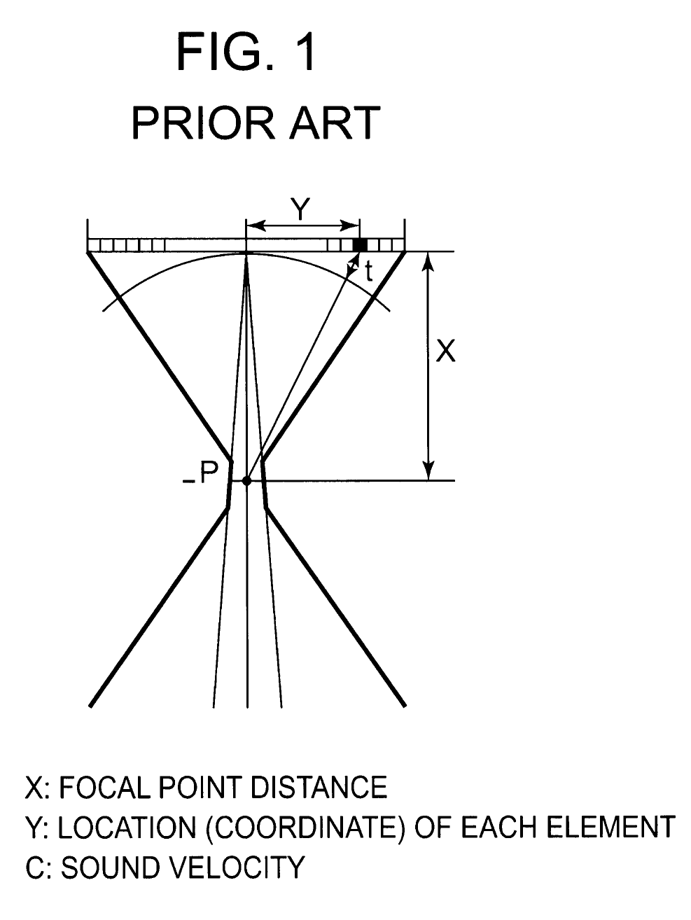

### DETAILED DESCRIPTION OF THE EMBODIMENTS

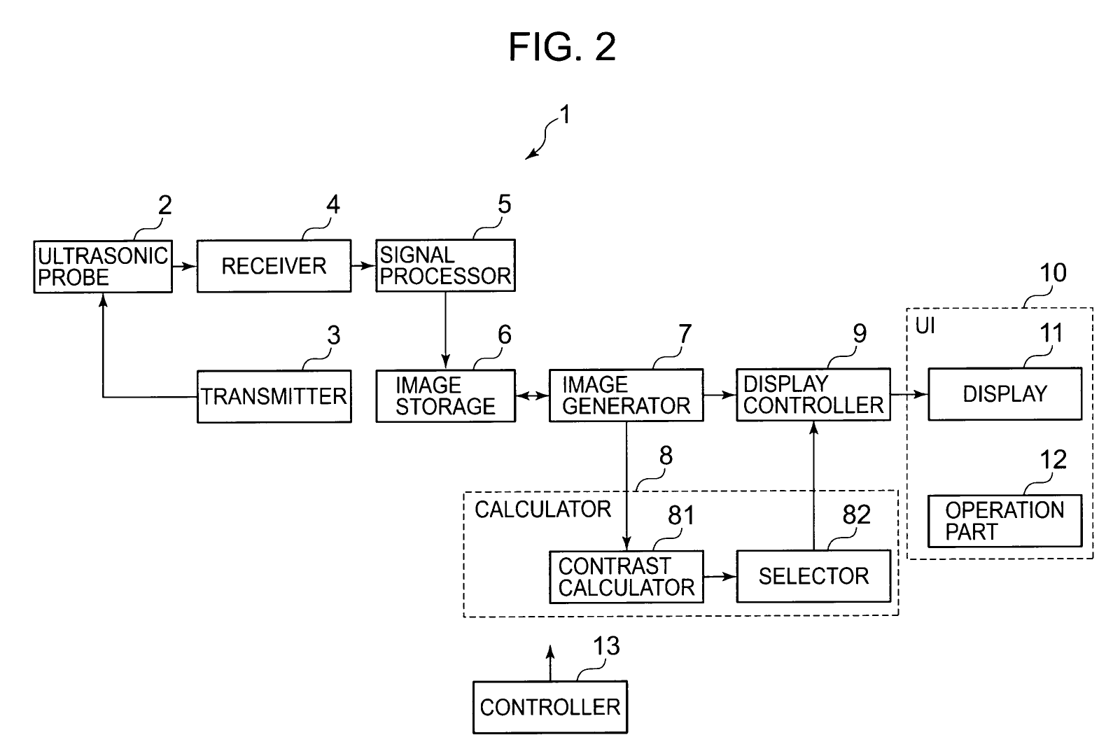

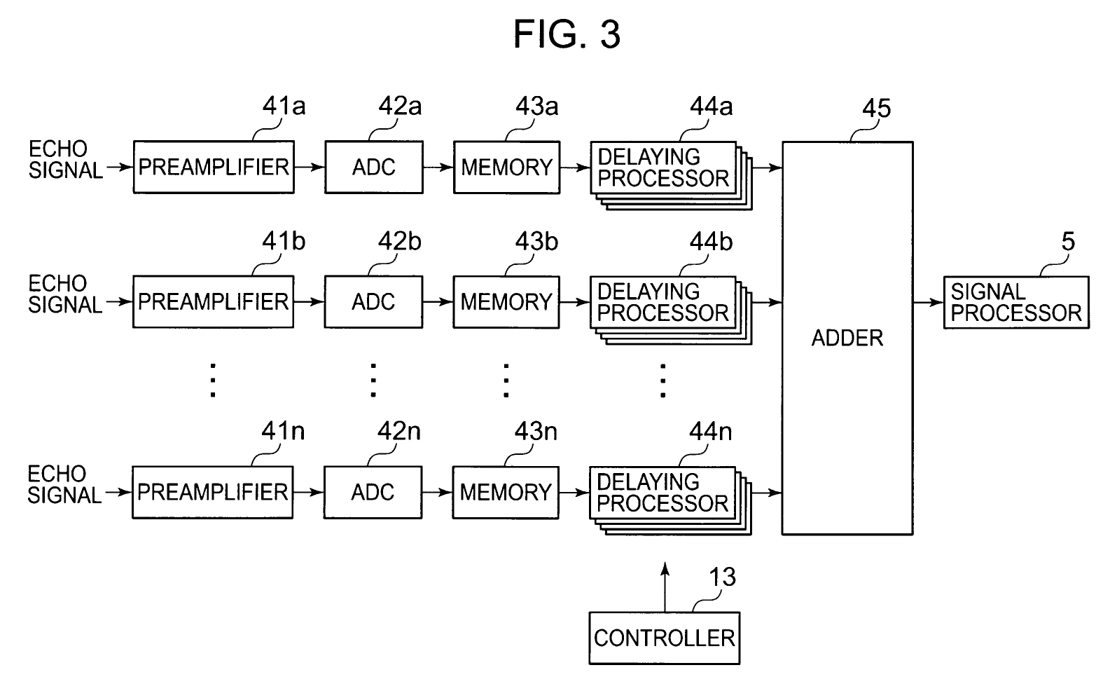

An ultrasonic imaging apparatus according to an embodiment of the present invention will be described with reference to FIG. 2 and FIG. 3. FIG. 2 is a block diagram showing the ultrasonic imaging apparatus according to the embodiment of the present invention. FIG. 3 is a block diagram showing a receiver installed in the ultrasonic imaging apparatus according to the embodiment of the present invention.

An ultrasonic imaging apparatus 1 according to the embodiment of the present invention comprises an ultrasonic probe 2, a transmitter 3, a receiver 4, a signal processor 5, an image storage 6, an image generator 7, a calculator 8, a display controller 9, a user interface (UI) 10, and a controller 13.

As the ultrasonic probe 2, a 1D array probe with a plurality of ultrasonic transducers aligned in a specified direction (a scanning direction) or a 2D array probe with a plurality of ultrasonic transducers arranged 2-dimensionally is used. The ultrasonic probe 2 transmits ultrasonic waves to an object, and receives reflected waves from the object as echo signals.

Under control of the controller 13, the transmitter 3 supplies electric signals to the ultrasonic probe 2 so as to transmit beamformed (transmission-beamformed) ultrasonic waves to a specified focal point.

The configuration of the transmitter 3 will be described in detail.

The transmitter includes a clock generation circuit, a transmission delay circuit, and a pulsar circuit, which are not shown in the drawings. The clock generation circuit generates a clock signal that determines the timing and frequency of transmission of an ultrasonic signal. The transmission delay circuit executes transmission focus by applying a delay at the time of transmission of ultrasonic waves. The pulsar circuit has the same number of pulsars as individual channels corresponding to the respective ultrasonic transducers. The pulsar circuit generates a driving pulse at delayed transmission timing, and supplies electric signals to the respective ultrasonic transducers of the ultrasonic probe 2.

The receiver 4 receives echo signals received by the ultrasonic probe 2 and executes a delaying process on the echo signals. By the delaying process, the receiver 4 converts the analog reception signals into reception-beamformed digital reception data and outputs to the signal processor 4. In other words, the receiver 4 adds the echo signals received at different times depending on a distance between a target reflector and each of the ultrasonic transducers in a state where the phases (times) of the echo signals are matched, thereby generating one line of focused reception data (signals for an image on one scanning line).

In this embodiment, the receiver 4 executes a delaying process in accordance with a plurality of set sound velocities to generate a plurality of reception data with different set sound velocities. For example, four types of set sound velocities are set in the receiver 4 in advance. The receiver 4 executes the delaying process in accordance with the four types of set sound velocities, respectively, thereby generating four types of reception data whose set sound velocities are different from each other. To be specific, the receiver 4 executes the delaying process while changing the value of the sound velocity C in the abovementioned formula (1), thereby generating four types of reception beams.

The specific configuration of the receiver 4 will be described with reference to FIG. 3. The receiver 4 includes: preamplifiers 41a, 41b . . . 41n (hereinafter, may be referred to as the “preamplifier 41a, etc.” representing individually); ADCs 42a, 42b . . . 42n (hereinafter, may be referred to as the “ADC 42a, etc.” representing individually), which are AD converters; memories 43a, 43b . . . 43n (hereinafter, may be referred to as the “memory 43a, etc.” representing individually); delaying processors 44a, 44b . . . 44n (hereinafter, may be referred to as the “delaying processor 44a, etc.” representing individually); and an adder 45.

The preamplifier 41a, etc., amplifies echo signals outputted from the respective ultrasonic transducers of the ultrasonic probe 2 at each of reception channels. Hereinafter, a signal line from each of the ultrasonic transducers may be referred to as a “channel.” The ADC 42a, etc., receives analog echo signals amplified by the preamplifier 41a, etc., and converts them into digital data in accordance with the accuracy of certain quantization. The echo signals converted into the digital data are once stored into the memory 43a, etc.

The delaying processor 44a, etc., reads out the echo signals stored in the memory 43a, etc., from the memory 43a, etc., in accordance with a delay time. Phase control (delay time control) on the reading-out timing depending on the distance between the focal point and each of the ultrasonic transducers makes it possible to match the phases of the respective echo signals. Then, the adder 45 adds the phase-matched echo signals from the plurality of channels, thereby generating a reception beam. The adder 45 then outputs the generated reception beams to the signal processor 5.

In this embodiment, for example, the memory 43a is provided with four delaying processors 44a, the memory 43b is provided with four delaying processors 44b . . . and the memory 43n is provided with four delaying processors 44n. The four delaying processors execute the delaying process in accordance with different set sound velocities.

Then, the adder 45 adds the echo signals having been subjected to the delaying process at the same set sound velocity, thereby generating a reception beam at the set sound velocity.

For example, among the four delaying processors 44a, etc., a first delaying processor 44a, etc. executes the delaying process in accordance with a first set sound velocity C1. The adder 45 adds the echo signals having been subjected to the delaying process in accordance with the first set sound velocity C1, thereby generating first reception data. A second delaying processor 44a, etc., executes the delaying process in accordance with a second set sound velocity C2.

The adder 45 adds the echo signals having been subjected to the delaying process in accordance with the second set sound velocity C2, thereby generating second reception data. A third delaying processor 44a, etc., executes the delaying process in accordance with a third set sound velocity C3. The adder 45 adds the echo signals having been subjected to the delaying process in accordance with the third set sound velocity C3, thereby generating third reception data. A fourth delaying processor 44a, etc., executes the delaying process in accordance with a fourth set sound velocity C4. The adder 45 adds the echo signals having subjected to the delaying process in accordance with the fourth set sound velocity C4, thereby generating fourth reception data. The first, second, third and fourth set sound velocities C1, C2, C3 and C4 have values different from each other, and are set in the controller 13 in advance. Under control of the controller 13, the four delaying processor 44a, etc., executes the delaying process in accordance with the four types of set sound velocities, respectively, thereby generating four types of reception data whose set sound velocities are different from each other.

For example, assuming the first set sound velocity C1 is 1,460 [m/s], the second set sound velocity C2 is 1,500 [m/s], the third set sound velocity C3 is 1,540 [m/s], and the fourth set sound velocity C4 is 1,580 [m/s], the four delaying processors 44a, etc., execute the delaying process in accordance with these sound velocities, respectively. The values of these set sound velocities can be changed arbitrarily by the operator. For example, when the operator inputs the value of a desired set sound velocity by using an operation part 12, the controller 13 sets the inputted value of the set sound velocity in the delaying processors 44a, etc.

As described above, the receiver 4 executes reception beamforming by changing the value of the set sound velocity to generate four types of reception beams.

Moreover, the receiver 4 may execute processing in accordance with parallel signal processing. For example, the receiver 4 may generate reception data in four different focal points existing around a certain focal point. In this case, the receiver 4 executes beamforming on the reception data of four directions while changing the set sound velocity for the delaying process. Consequently, the receiver 4 simultaneously generates the same number of reception beams as obtained by multiplying the number of the reception data of four directions by the number of the set sound velocities. For example, in the case of executing the delaying process in accordance with four types of set sound velocities, the receiver 4 simultaneously generates sixteen reception beams obtained from (four directions)×(four types of set sound velocities). In this case, by providing the memory 43a with sixteen delaying processors 44a, the memory 43b with sixteen delaying processors 44b . . . and the memory 43n with sixteen delaying processors 44n, sixteen reception beams are simultaneously generated.

The signal processor 5 includes a B-mode processor. The B-mode processor images amplitude information of the echoes and generates B-mode ultrasound raster data from reception data. To be specific, the B-mode processor executes a Band Pass Filter process on the reception data outputted from the receiver 4, and thereafter detects the envelope curve of outputted signals. Then, the B-mode processor images the amplitude information of the echoes by executing a compression process using logarithmic transformation on the detected data.

The signal processor 5 may include a Doppler processor. For example, the Doppler processor executes quadrature detection on the reception signals sent from the transmitter 3 to extract Doppler shift frequency components, and further executes an FFT (Fast Fourier Transform) process to generate a Doppler frequency distribution representing a blood flow velocity.

The signal processor 5 may include a color mode processor. The color mode processor images information on a moving blood flow by generating color ultrasound raster data. The blood-flow information includes information such as the velocity, diffusion, and power. The blood-flow information is obtained as binary information.

The reception data outputted from the receiver 4 is processed in either of the processors.

The signal processor 5 outputs the ultrasound raster data to the image storage 6. The image storage 6 stores the ultrasound raster data.

In this embodiment, the signal processor 5 receives a plurality of reception beams with different set sound velocities from the receiver 4, and generates a plurality of B-mode ultrasound raster data with different set sound velocities. For example, in a case where reception beams are generated in accordance with the four types of set sound velocities (C1, C2, C3 and C4), the signal processor 5 processes the reception beams generated in accordance with the respective set sound velocities to generate B-mode ultrasound raster data corresponding to the respective set sound velocities.

In order to generate an ultrasonic image, the ultrasonic probe 2, the transmitter 3 and the receiver 4 scan a desired imaging region with ultrasonic waves to generate scan line signals (reception data) for one screen (one frame). Then, the ultrasound raster data generated in the B-mode processor of the signal processor 5 is stored into the image storage 6. For example, in a case where one frame is composed of 380 lines of scan line signals, the same number of reception data as obtained by multiplying 380 by the number of set sound velocities are generated and stored into the image storage 6.

The image generator 7 generates image data based on the ultrasound raster data stored in the image storage 6. For example, the image generator 7 includes a DSC (Digital Scan Converter), which converts the ultrasound raster data into image data represented by orthogonal coordinates (a scan conversion process). For example, the DSC generates tomographic image data as 2-dimensional information based on the B-mode ultrasound raster data.

In this embodiment, the image generator 7 generates a plurality of tomographic image data with different set sound velocities, based on a plurality of B-mode ultrasound raster data with different set sound velocities. For example, in a case where reception beams are generated in accordance with the four types of set sound velocities (C1, C2, C3 and C4), the image generator 7 generates four types of tomographic image data with different set sound velocities. Then, the image generator 7 outputs the four types of tomographic image data with different set sound velocities to the calculator 8.

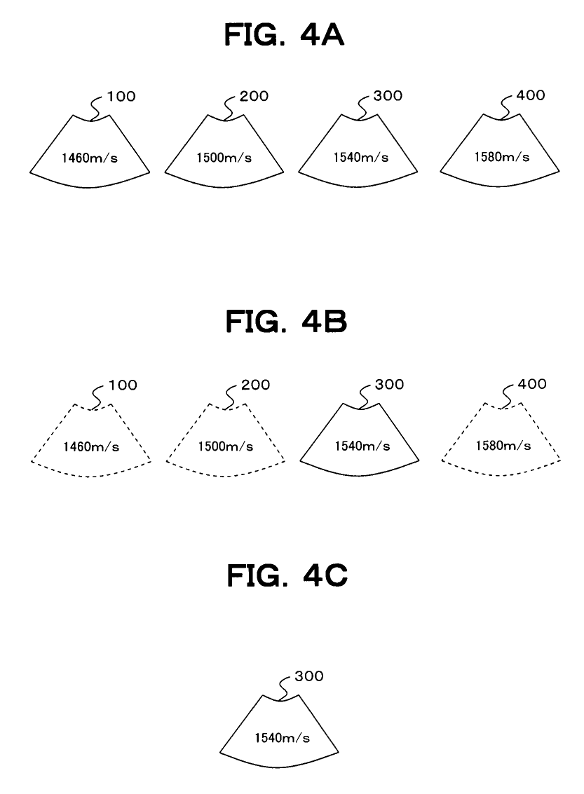

Tomographic images generated by the image generator 7 will be described with reference to FIGS. 4A-4C. FIG. 4A is a view schematically showing tomographic images generated in accordance with different set sound velocities. FIG. 4B is a view schematically showing tomographic images generated in accordance with different set sound velocities. FIG. 4C is a view schematically showing a tomographic image.

In this embodiment, the delaying process is executed in accordance with the four types of set sound velocities, so that four types of tomographic images are generated. For example, as shown in FIG. 4A, a tomographic image 100 is an image generated under the condition of the set sound velocity=1,460 [m/s]. A tomographic image 200 is an image generated under the condition of the set sound velocity=1,500 [m/s]. A tomographic image 300 is an image generated under the condition of the set sound velocity=1,540 [m/s]. A tomographic image 400 is an image generated under the condition of the set sound velocity=1,580 [m/s].

The calculator 8 includes a contrast calculator 81 and a selector 82. The contrast calculator 81 receives a plurality of tomographic image data with different set sound velocities from the image generator 7, and obtains the ratio of light and dark (i.e., the contrast) of each of the tomographic images. For example, the contrast calculator 81 obtains the dispersion value of luminance of a tomographic image, the rate of change in luminance of a tomographic image, or the like as the contrast. In this embodiment, four types of tomographic image data are generated in accordance with the four set sound velocities, so that the contrast calculator 81 obtains the contrast of each of the four types of tomographic image data.

The selector 82 selects tomographic image data with the highest contrast from among the plurality of tomographic image data, and outputs the selected tomographic image data to the display controller 9.

For example, the selector 82 selects a tomographic image with the largest dispersion value of luminance as the tomographic image with the highest contrast. Alternatively, the selector 82 may select a tomographic image with the highest rate of change in luminance in the tomographic image as the tomographic image with the highest contrast.

The tomographic image with the highest contrast is presumed to be an image in which the set sound velocity and living-body sound velocity are the closest. If the living-body sound velocity and set sound velocity are equal, the resolution is high, and the contrast in the ultrasonic image is high. Conversely, if the living-body sound velocity and set sound velocity are different from each other, the resolution is low, and the contrast in the ultrasonic image is low. Therefore, by selecting the tomographic image with the highest contrast from among the plurality of tomographic images, a tomographic image generated under the condition in which the set sound velocity is the closest to the living-body sound velocity is selected.

For example, as shown in FIG. 4B, if the contrast of the tomographic image 300 is the highest among the tomographic images 100, 200, 300 and 400, the selector 82 selects the tomographic image 300 and outputs tomographic image data thereof to the display controller 9.

The display controller 9 controls a display 11 to display a tomographic image based on the tomographic image data outputted from the calculator 8. Thus, a tomographic image with the highest contrast is displayed on the display 11. For example, as shown in FIG. 4C, the display controller 9 controls the display 11 to display the tomographic image 300.

The user interface (UI) 10 includes the display 11 and the operation part 12. The display 11 is composed of a monitor such as a CRT and a liquid crystal display, and displays a tomographic image, etc.

The operation part 12 is composed of a pointing device such as a joystick and a trackball, a switch, various kinds of buttons, a mouse, a keyboard, a TCS (Touch Command Screen), or the like.

The controller 13 is connected to each part of the ultrasonic imaging apparatus 1, and controls the operation of each part of the ultrasonic imaging apparatus 1. For example, the controller 13 includes an information processing device such as a CPU (Central Processing Unit), and a storage device such as a ROM (Read Only Memory) and a RAM (Random Access Memory), which are not shown in the drawings.

The information processing device executes a control program, thereby controlling the operation of each part of the ultrasonic imaging apparatus 1.

The calculator 8 includes a CPU and a storage device such as a ROM, a RAM and an HDD (Hard Disk Drive), which are not shown in the drawings. The storage device stores a calculation program for executing a function of the calculator 8. This calculation program includes a contrast-calculation program for executing the function of the contrast calculator 81 and a selecting program for executing the function of the selector 82. The CPU executes the contrast-calculation program, thereby obtaining the contrast of the tomographic image data.

Moreover, the CPU executes the selecting program, thereby selecting tomographic image data with the highest contrast.

Further, the display controller 9 includes a CPU and a storage device such as a ROM, a RAM and a HDD, which are not shown in the drawings. A display control program for executing the function of the display controller 9 is stored in the storage device. The CPU executes the display control program, thereby controlling the display 11 to display an ultrasonic image.

### (Operation)

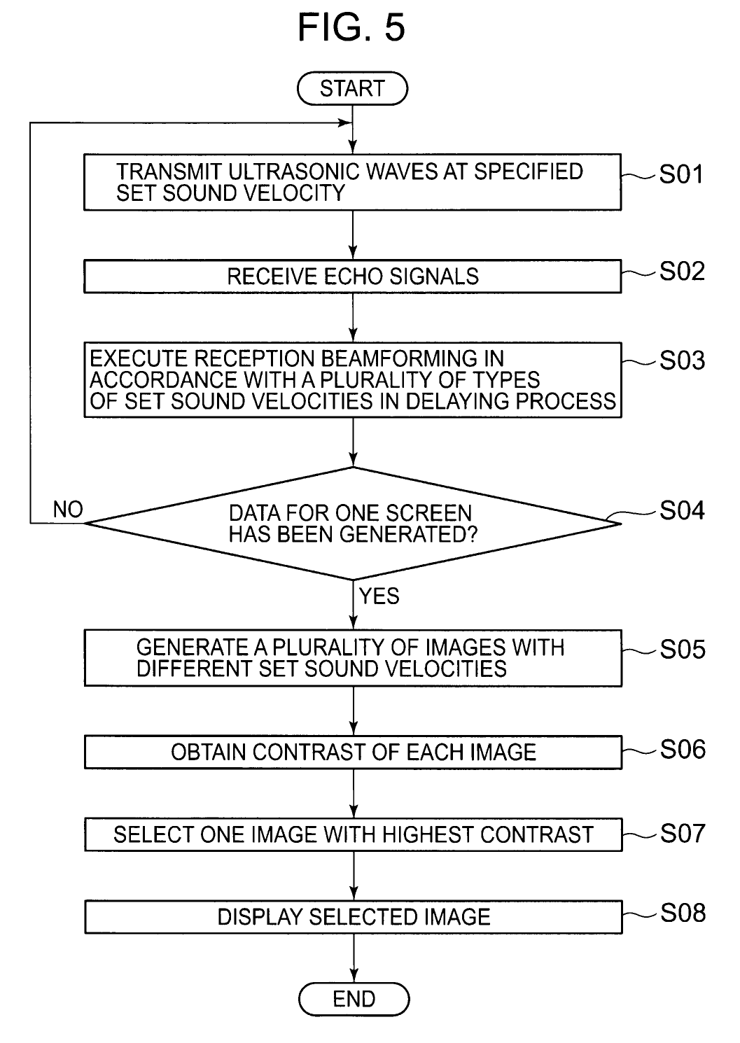

Next, a series of operations by the ultrasonic imaging apparatus according to the embodiment of the present invention will be described with reference to FIG. 5. FIG. 5 is a flow chart showing the series of operations by the ultrasonic imaging apparatus according to the embodiment of the present invention.

### (Step S01)

First, the transmitter 3 transmits ultrasonic waves to an object by the ultrasonic probe 2 at a specified set sound velocity.

### (Step S02)

The ultrasonic probe 2 receives echo signals reflected from the object and outputs the echo signals to the receiver 4.

### (Step S03)

The receiver 4 executes a delaying process on the echo signals outputted from the ultrasonic probe 2 in accordance with different set sound velocities, thereby generating a plurality of reception data with different set sound velocities. For example, in accordance with the first set sound velocity C1 of 1,460 [m/s], the second set sound velocity C2 of 1,500 [m/s], the third set sound velocity C3 of 1,540 [m/s] and the fourth set sound velocity C4 of 1,580 [m/s], the receiver 4 executes reception beamforming while changing the value of the set sound velocity, thereby generating four types of reception beams. Then, the signal processor 5 receives the plurality of reception beams with different set sound velocities from the receiver 4, and generates a plurality of B-mode ultrasound raster data with different set sound velocities. The B-mode ultrasound raster data are stored into the image storage 6.

(Step S04) The process from Step S01 to Step S03 is repeated until data for one screen (one frame) is generated and stored into the image storage 6.

Consequently, the same number of data as obtained by multiplying the data for one screen (one frame) by the number of set sound velocities are generated and stored into the image storage 6. For example, in a case where one frame is composed of 380 lines of scan line signals, the process from Step S01 to Step S03 is repeated until the same number of reception data as obtained by multiplying 380 by the number of set sound velocities (e.g., 4) are generated and stored into the image storage 6.

### (Step S05)

When the data for one frame is generated and stored into the image storage 6 (Step S04, Yes), the image generator 7 reads the plurality of B-mode ultrasound raster data with different set sound velocities from the image storage 6, and generates a plurality of tomographic image data with different set sound velocities. For example, in a case where the reception beams are generated in accordance with the four set sound velocities (C1, C2, C3 and C4), the image generator 7 generates four types of tomographic images 100, 200, 300 and 400 with different set sound velocities as shown in FIG. 4A.

The image generator 7 then outputs the four types of tomographic image data to the calculator 8.

### (Step S06)

The contrast calculator 81 obtains the contrast of each of the plurality of tomographic image data with different set sound velocities.

For example, the contrast calculator 81 obtains, as contrast, the dispersion value of luminance of the tomographic image, the rate of change in luminance of the tomographic image, or the like.

### (Step S07)

Then, the selector 82 selects tomographic image data with the highest contrast from among the plurality of tomographic image data with different set sound velocities, and outputs the selected tomographic image data to the display controller 9. For example, the selector 82 selects a tomographic image with the highest dispersion value of luminance as a tomographic image with the highest contrast.

Alternatively, the selector 82 may select a tomographic image with the highest rate of change in luminance in the tomographic image as the tomographic image with the highest contrast. For example, as shown in FIG. 4B, in a case where the tomographic image 300 has the highest contrast among the tomographic images 100, 200, 300 and 400, the selector 82 selects the tomographic image 300 and outputs the tomographic image data thereof to the display controller 9. Thus, by selecting a tomographic image with the highest contrast, a tomographic image generated under the condition that the set sound velocity is the closest to the living-body sound velocity is selected.

### (Step S08)

The display controller 9 receives the tomographic image data from the selector 82 and controls the display 11 to display a tomographic image based on the tomographic image data. For example, as shown in FIG. 4C, the display controller 9 controls the display 11 to display the tomographic image 300 with the highest contrast. Thus, only the tomographic image 300 generated under the condition that the set sound velocity is 1,540 [m/s] is displayed on the display 11.

As described above, it is possible to generate tomographic image data with different set sound velocities by executing the delaying process in accordance with a plurality of set sound velocities, and provide a high-resolution tomographic image by displaying a tomographic image based on tomographic image data with the highest contrast from among the plurality of tomographic image data. Moreover, since it is not necessary to scan for checking a sound velocity unlike in the related art, it is possible to optimize a set sound velocity in real time at the time of imaging for diagnosis, and obtain a high-resolution tomographic image. Moreover, since it is not necessary to scan for checking a set sound velocity, it is possible to obtain a high-resolution tomographic image without scanning for check even if an imaging position is displaced. Thus, there is no need to scan for check repeatedly, and therefore, complicatedness in operation is eliminated. a result, it is possible to shorten the duration for diagnosis.

Further, the receiver 4 may execute a process by parallel signal processing. To be specific, the receiver 4 executes beamforming on reception beams of a plurality of directions while changing the set sound velocity for a delaying process. Consequently, the receiver 4 simultaneously generates the same number of reception beams as obtained by multiplying the number of reception beams of a plurality of directions by the number of set sound velocities. For example, the receiver 4 may execute the delaying process on reception beams of four directions in accordance with four set sound velocities, thereby simultaneously generating sixteen lines of reception beams.

In this embodiment, four types of tomographic image data are generated in accordance with four set sound velocities. This is one example, and the delaying process may be executed in accordance with any number of set sound velocities other than four. For example, the delaying process may be executed in accordance with five or more set sound velocities, or the delaying process may be executed in accordance with two or three set sound velocities.

Moreover, when tomographic image data is selected by the selector 82, the controller 13 may newly obtain a plurality of set sound velocities with reference to a set sound velocity for generating the selected tomographic image data. For example, with reference to the selected set sound velocity, the controller 13 obtains a plurality of set sound velocities by changing a sound velocity by a specified value.

When echo signals are newly received by the new scan, the delaying processor 44a, etc., executes the delaying process on the new echo signals in accordance with a plurality of newly obtained set sound velocities, thereby generating a plurality of reception data with different set sound velocities.

If tomographic image data generated under the condition that a set sound velocity is 1,540 [m/s] is selected by the selector 82, the controller 13 obtains a plurality of sound velocities by changing the sound velocity by a specified value, with reference to the set sound velocity of 1,540 [m/s]. For example, with reference to the set sound velocity of 1,540 [m/s], the controller 13 obtains a plurality of set sound velocities by changing the sound velocity by 40 [m/s].

In a case where the delaying process is executed in accordance with four set sound velocities, the controller 13 obtains a first set sound velocity (1,500 [m/s]), a second set sound velocity (1,540 [m/s]), a third set sound velocity (1,580 [m/s]) and a fourth set sound velocity (1,620 [m/s]) with reference to the set sound velocity of 1,540 [m/s], for example. Then, when echo signals are newly received by the new scan, the delaying processor 44a, etc., executes the delaying process on the new echo signals in accordance with the first set sound velocity (1,500 [m/s]), the second set sound velocity (1,540 [m/s]), the third set sound velocity (1,580 [m/s]) and the fourth set sound velocity (1,620 [m/s]).

Further, in a case where the delaying process is executed in accordance with five set sound velocities, the controller 13 sets the set sound velocity of 1,540 [m/s] as the center value and obtains a first set sound velocity (1,460 [m/s]), a second set sound velocity (1,500 [m/s]), a third set sound velocity (1,540 [m/s]), a fourth set sound velocity (1,580 [m/s]) and a fifth set sound velocity (1,620 [m/s]). Then, when echo signals are newly received by the new scan, the delaying processor 44a, etc., executes the delaying process on the new echo signals in accordance with the first set sound velocity (1,460 [m/s]), the second set sound velocity (1,500 [m/s]), the third set sound velocity (1,540 [m/s]), the fourth set sound velocity (1,580 [m/s]) and the fifth set sound velocity (1,620 [m/s]).

After that, every time new scan is executed and tomographic image data is selected by the selector 82, with reference to a selected set sound velocity, the controller 13 newly obtains a plurality of set sound velocities.

As described above, by newly obtaining a plurality of set sound velocities with reference to a selected set sound velocity, it becomes possible to acquire a more appropriate set sound velocity in real time to execute the delaying process.

### Modification

Next, modifications of the ultrasonic imaging apparatus 1 according to the abovementioned embodiment will be described.

### (Modification 1)

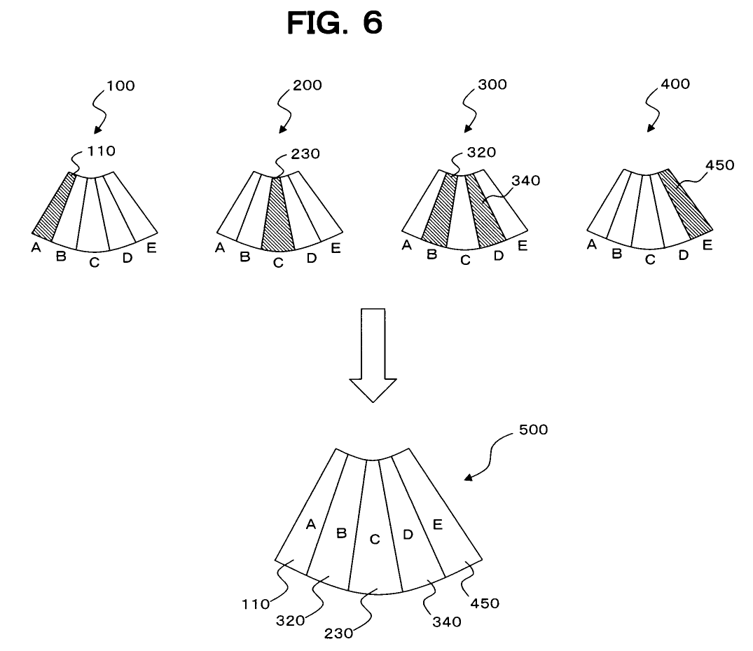

First, Modification 1 of the ultrasonic imaging apparatus 1 will be described with reference to FIG. 6. FIG. 6 is a schematic view showing tomographic images generated in accordance with different set sound velocities.

Since a living body has various tissue characterizations such as muscle and fat, the values of set sound velocities at which the resolution and contrast become high vary depending on sites. In Modification 1, each of a plurality of tomographic images with different set sound velocities is divided into a plurality of individual regions, and the contrast in each of the individual regions of the respective tomographic images is obtained. Then, from among the plurality of tomographic images with different set sound velocities, tomographic image data with the highest contrast is selected for each of the individual regions. By coupling the tomographic image data with the highest contrast for the respective individual regions, tomographic image data representing the entire region is reconstructed.

Consequently, a high-resolution tomographic image is obtained even if the set sound velocity at which the contrast becomes high varies depending on the individual region of the tomographic image, because a tomographic image with the highest contrast is selected for each of the individual regions from among the plurality of tomographic images with different set sound velocities. A specific process will be described below.

In Modification 1, the contrast calculator 81 divides each of a plurality of tomographic images with different set sound velocities into a plurality of individual regions, and obtains the contrast in each of the individual regions of the respective tomographic images. For example, as shown in FIG. 6, the contrast calculator 81 divides the tomographic image 100 generated under the condition of the first set sound velocity C1 into five individual regions A, B, C, D and E. Then, the contrast calculator 81 obtains the contrast in the tomographic image data for each of the individual regions A to E. In other words, in the tomographic image 100, the contrast calculator 81 obtains the contrast of the tomographic image data of the individual region A, the contrast of the tomographic image data of the individual region B, the contrast of the tomographic image data of the individual region C, the contrast of the tomographic image data of the individual region D, and the contrast of the tomographic image data of the individual region E.

Similarly, the contrast calculator 81 divides each of the tomographic images 200, 300 and 400 into five individual regions A to E, and obtains the contrast of the tomographic image data for each of the individual regions.

Information (coordinate information) showing a division pattern for dividing into individual regions is previously set in the controller 13. The contrast calculator 81 divides a tomographic image into a plurality of individual regions under the control of the controller 13. In the example shown in FIG. 6, a tomographic image is divided into a plurality of individual regions along the transmitting directions of ultrasonic waves. The division pattern shown in FIG. 6 is one example, and a tomographic image may be divided into a plurality of individual regions in accordance with a division pattern other than the pattern described above. Moreover, a tomographic image may be equally divided so that the respective individual regions are equal in size, or a tomographic image may be divided so that the respective individual regions are different in size. Furthermore, the operator may designate an arbitrary division pattern by using the operation part 12. Although the tomographic image is divided so that individual regions adjacent to each other do not overlap in the example shown in FIG. 6, the entire tomographic image may be divided so that the individual regions adjacent to each other overlap. When an arbitrary division pattern is designated by using the operation part 12, the controller 13 sets the designated division pattern in the contrast calculator 81. The contrast calculator 81 divides the tomographic image into a plurality of individual regions in accordance with the division pattern.

For the plurality of tomographic images with different set sound velocities, the selector 82 selects tomographic image data with the highest contrast from among the tomographic image data of the same individual regions. For example, when the contrast in the tomographic image 100 generated under the condition of the set sound velocity C1 is the highest of all the individual regions A, the selector 82 selects a tomographic image 110 of the individual region A. Similarly, when the contrast in the tomographic image 300 generated under the condition of the set sound velocity C3 is the highest of all the individual regions B, the selector 82 selects a tomographic image 320 of the individual region B. Moreover, when the contrast in the tomographic image 200 generated under the condition of set sound velocity C2 is the highest of all the individual regions C, the selector 82 selects a tomographic image 230 of the individual region C. Moreover, when the contrast in the tomographic image 300 generated under the condition of the set sound velocity C3 is the highest of all the individual regions D, the selector 82 selects a tomographic image 340 of the individual region D.

Moreover, when the contrast in the tomographic image 400 generated under the condition of set sound velocity C4 is the highest of all the individual regions E, the selector 82 selects a tomographic image 450 of the individual region E.

Then, the selector 82 outputs the tomographic image data with the highest contrast of each of the individual regions A through E to the display controller 9.

The display controller 9 couples the tomographic image data with the highest contrasts of the respective individual regions A through E, and reconstructs one tomographic image data. In the example shown in FIG. 6, the display controller 9 couples the tomographic image 110 of the individual region A, the tomographic image 320 of the individual region B, the tomographic image 230 of the individual region C, the tomographic image 340 of the individual region D, and the tomographic image 450 of the individual region E, thereby reconstructing one tomographic image 500.

The display controller 9 controls the display 11 to display the tomographic image 500 based on the reconstructed tomographic image data on the display 11. Thus, even if the set sound velocity with the high contrast varies depending on regions in tomographic images, a tomographic image with the highest contrast is selected for each of the regions. Therefore, a tomographic image with high resolution on the whole is obtained.

### (Operation)

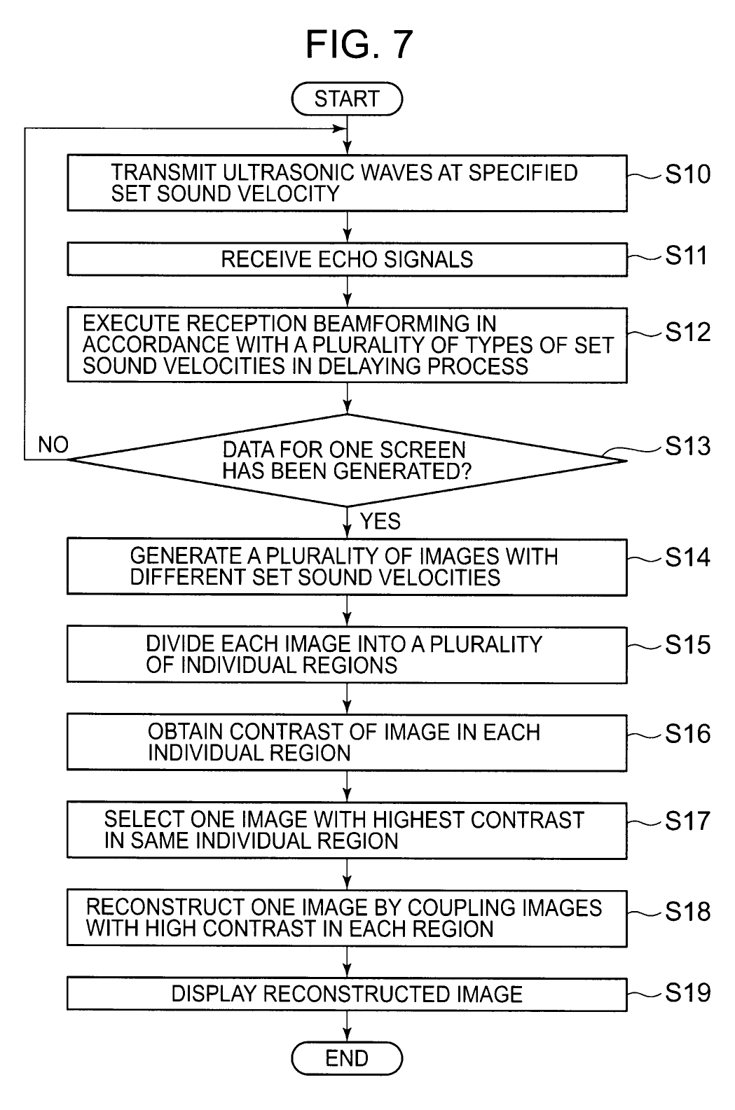

Next, a series of operations by the ultrasonic imaging apparatus according to Modification 1 will be described with reference to FIG. 7. FIG. 7 is a flow chart showing the series of operations by the ultrasonic imaging apparatus according to Modification 1.

### (Step S10)

First, the transmitter 3 transmits ultrasonic waves to an object by the ultrasonic probe 2 at a specified set sound velocity.

### (Step S11)

The ultrasonic probe 2 receives echo signals reflected from the object and outputs the echo signals to the receiver 4.

### (Step S12)

The receiver 4 executes a delaying process on the echo signals outputted from the ultrasonic probe 2 in accordance with different set sound velocities, thereby generating reception data with different set sound velocities. For example, in accordance with a first set sound velocity C1, a second set sound velocity C2, a third set sound velocity C3 and a fourth set sound velocity C4, the receiver 4 executes reception beamforming while changing the value of the set sound velocity, thereby generating four types of reception data. The signal processor 5 then receives the plurality of reception data with different set sound velocities and generates a plurality of B-mode ultrasound raster data with different set sound velocities. These B-mode ultrasound raster data are stored into the image storage 6.

### (Step S13)

Then, the process from Step S11 to Step S12 is repeated until data for one screen (one frame) is generated and stored into the image storage 6. Consequently, the same number of data as obtained by multiplying the data for one screen (one frame) by the number of set sound velocities are generated and stored into the image storage 6. For example, in a case where one frame is composed of 380 lines of scan line signals, the process from Step S10 to Step S12 is repeated until the same number of reception data as obtained by multiplying 380 by the number of set sound velocities (e.g., 4) are generated and stored into the image storage 6.

### (Step S14)

Then, when the data for one frame is generated and stored into the image storage 6 (Step S13, Yes), the image generator 7 reads the plurality of B-mode ultrasound raster data with different set sound velocities from the image storage 6 and generates a plurality of tomographic image data with different set sound velocities. For example, in a case where reception beams are generated in accordance with the four set sound velocities (C1, C2, C3 and C4), the image generator 7 generates four types of tomographic images 100, 200, 300 and 400 with different set sound velocities as shown in FIG. 6. The image generator 7 then outputs the four types of tomographic image data to the calculator 8.

### (Step S15)

The contrast calculator 81 divides each of the tomographic images generated under the conditions of the different set sound velocities into a plurality of individual regions. For example, as shown in FIG. 6, the contrast calculator 81 divides the tomographic image 100 generated under the condition of the set sound velocity C1 into five individual regions A, B, C, D and E. Similarly, the contrast calculator divides each of the tomographic image 200 generated under the condition of the set sound velocity C2, the tomographic image 300 generated under the condition of the set sound velocity C3 and the tomographic image 400 generated under the condition of the set sound velocity C4, into the five individual regions A through E.

### (Step S16)

Then, the contrast calculator 81 obtains the contrast of each of the tomographic image data of the respective individual regions. In the example shown in FIG. 6, for the tomographic image 100, the contrast calculator 81 obtains the contrast of the tomographic image data of the individual region A, the contrast of the tomographic image data of the individual region B, the contrast of the tomographic image data of the individual region C, the contrast of the tomographic image data of the individual region D, and the contrast of the tomographic image data of the individual region E. Similarly, for the tomographic images 200, 300 and 400, the contrast calculator 81 obtains the contrasts of the tomographic image data of the individual regions A through E.

### (Step S17)

The selector 82 selects one tomographic image with the highest contrast in the same individual regions from among the plurality of tomographic images with different set sound velocities. In the example shown in FIG. 6, the selector 82 selects: the tomographic image 110 generated in accordance with the set sound velocity C1 for the individual region A; the tomographic image 320 generated in accordance with the set sound velocity C3 for the individual region B; the tomographic image 230 generated in accordance with the set sound velocity C2 for the individual region C; the tomographic image 340 generated in accordance with the set sound velocity C3 for the individual region D; and the tomographic image 450 generated in accordance with the set sound velocity C4 for the individual region E.

### (Step S18)

The display controller 9 couples the tomographic image data with the highest contrast in the respective individual regions A through E, thereby reconstructing one tomographic image data. In the example shown in FIG. 6, the display controller 9 couples the tomographic image 110 of the individual region A, the tomographic image 320 of the individual region B, the tomographic image 230 of the individual region C, the tomographic image 340 of the individual region D, and the tomographic image 450 of the individual region E, thereby reconstructing a single tomographic image 500.

### (Step S19)

The display controller 9 then controls the display 11 to display the tomographic image 500 based on the reconstructed tomographic image data.

As described above, by dividing each of the tomographic images generated under the conditions of the respective set sound velocities into a plurality of individual regions and selecting a tomographic image with the highest contrast for each of the individual regions, it is possible to acquire a high-resolution tomographic image as a whole even if the set sound velocity at which the contrast becomes high varies depending on regions in the tomographic image.

### (Modification 2)

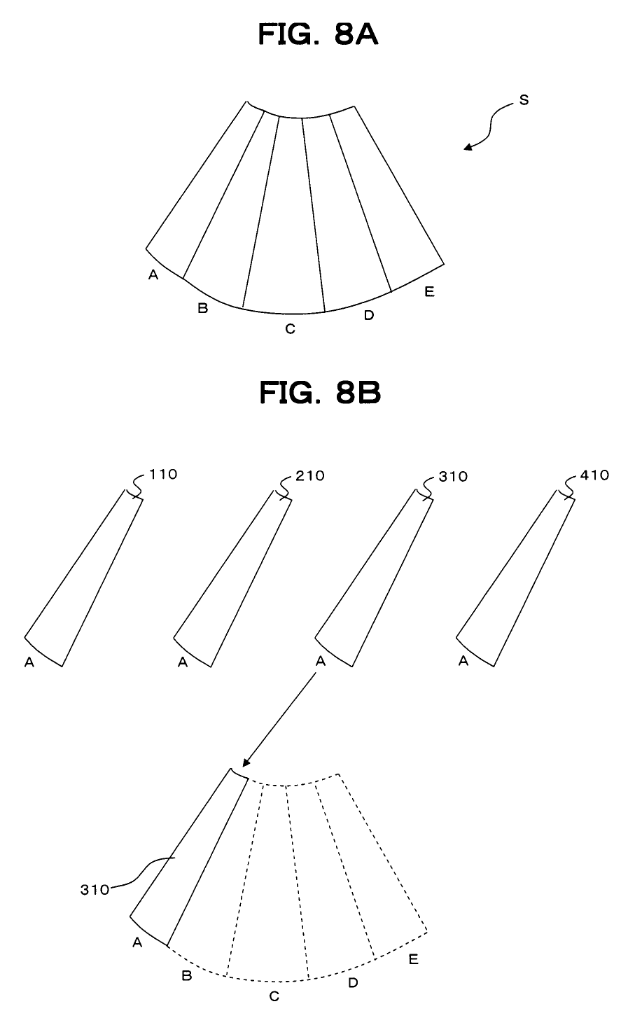

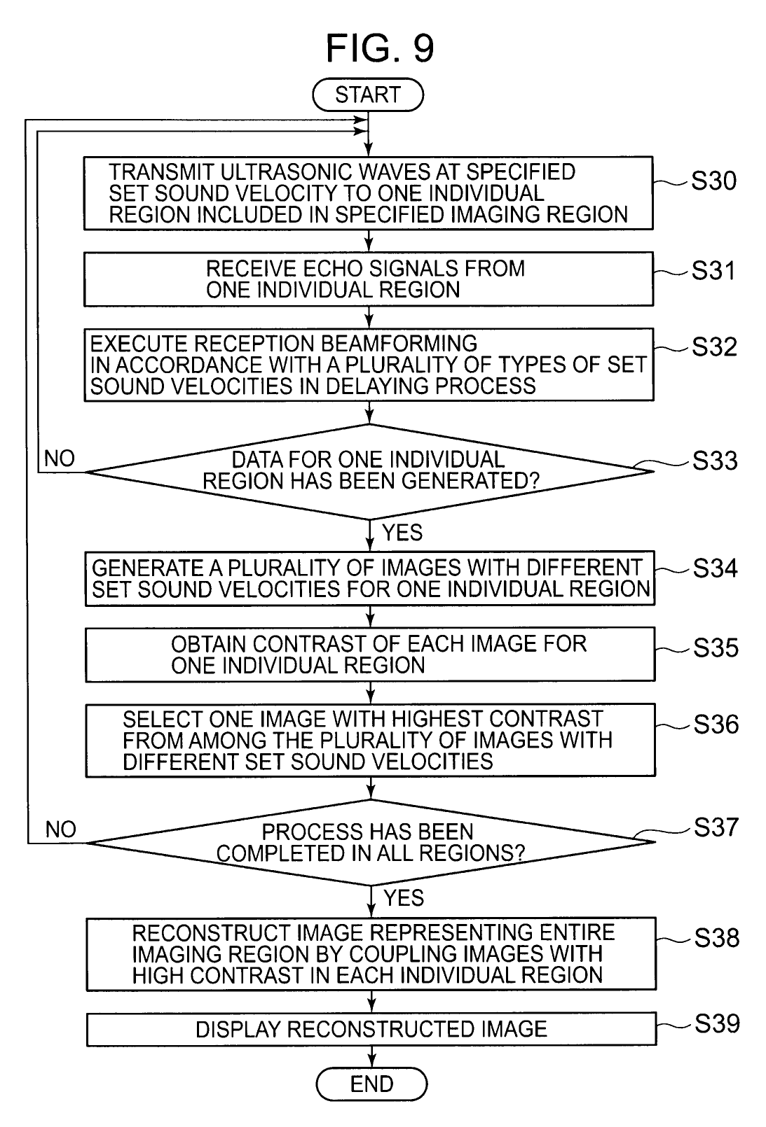

Next, Modification 2 of the ultrasonic imaging apparatus 1 will be described with reference to FIG. 8A, FIG. 8B and FIG. 9. FIG. 8A is a view schematically showing an imaging region. FIG. 8B is a view schematically showing tomographic images in the imaging region. FIG. 9 is a flow chart showing a series of operations by the ultrasonic imaging apparatus according to Modification 2.

In Modification 2, an entire imaging region is divided into a plurality of individual regions, and transmission/reception of ultrasonic waves, generation of tomographic image data, calculation of contrast, and selection of tomographic image data are executed for each of the individual regions. Data that has not been selected is deleted from the image storage 6 after every selection. The operation of the ultrasonic imaging apparatus according to Modification 2 will be described below with reference to the flow chart shown in FIG. 9.

### (Step S30)

First, the transmitter 3 divides a desired imaging region into a plurality of individual regions under the control of the controller 13, and transmits ultrasonic waves to one of the individual regions at a specified set sound velocity. For example, as shown in FIG. 8A, the transmitter 3 divides an entire imaging region S into a plurality of individual regions A, B, C, D and E, and sequentially transmits ultrasonic waves to each of the individual regions. Information (coordinate information) indicating the entire imaging region S and information (coordinate information) indicating the respective individual regions A through E are set in the controller 13 in advance. The transmitter 3 then transmits ultrasonic waves to one of the individual regions under the control of the controller 13.

### (Step S31)

The ultrasonic probe 2 receives echo signals reflected from one of the individual regions included in the entire imaging region S, and outputs the echo signals to the receiver 4. For example, the ultrasonic probe 2 receives echo signals reflected from the individual region A, and outputs the echo signals of the individual region A to the receiver 4.

### (Step S32)

The receiver 4 executes a delaying process on the echo signals from one of the individual regions outputted from the ultrasonic probe 2 in accordance with different set sound velocities, thereby generating a plurality of reception data with different set sound velocities. For example, when ultrasonic waves are transmitted to the individual region A, the receiver 4 executes the delaying process on the echo signals from the individual region A in accordance with the different set sound velocities under the control of the controller 13, thereby generating a plurality of reception data with different set sound velocities. For example, in accordance with the first set sound velocity C1, the second set sound velocity C2, the third set sound velocity C3 and the fourth set sound velocity C4, the receiver 4 executes reception beamforming while changing the value of the set sound velocity, thereby generating four types of reception data in the individual region A. The signal processor 5 then receives the plurality of reception data of the individual region A, and generates a plurality of B-mode ultrasound raster data with different set sound velocities. The plurality of B-mode ultrasound raster data are temporarily stored into the image storage 6.

### (Step S33)

The process from Step S30 to Step S32 is repeated until data for one of the individual regions is generated and stored into the image storage 6. By repeating transmission/reception of ultrasonic waves to/from one of the individual regions, data of the individual region is acquired. Consequently, the same number of data as obtained by multiplying the number of data of one of the individual regions by the number of the set sound velocities are generated and stored into the image storage 6. For example, in a case where one frame is composed of 380 lines of scan line signals, division of 380 by the number of the individual regions (e.g., 5) is executed. Then, the process from Step 30 to Step 32 is repeated until the same number of reception data as obtained by multiplying the number obtained by the division by the number of the set sound velocities (e.g., 4) are generated and stored in the image storage 6.

### (Step S34)

When data for one of the individual regions is generated and stored into the image storage 6 (Step S33, Yes), the image generator 7 reads the plurality of B-mode ultrasound raster data with different set sound velocities from the image storage 6, and generates a plurality of tomographic image data with different set sound velocities. For example, in a case where ultrasonic waves are transmitted to the individual region A, the image generator 7 reads a plurality of B-mode ultrasound raster data with different set sound velocities of the individual region A from the image storage 6, and generates a plurality of tomographic image data with different set sound velocities in the individual region A. For example, as shown in FIG. 8B, in the individual region A, the image generator 7 generates a tomographic image 110 under the condition of the set sound velocity C1, a tomographic image 210 under the condition of the set sound velocity C2, a tomographic image 310 under the condition of the set sound velocity C3, and a tomographic image 410 under the condition of the set sound velocity C4.

### (Step S35)

For one of the individual regions, the contrast calculator 81 obtains the contrasts of tomographic images generated under the conditions of different set sound velocities. In the example shown in FIG. 8B, the contrast calculator 81 obtains the contrasts of the tomographic images 110, 210, 310 and 410 in the individual region A.

### (Step S36)

For one of the individual regions, the selector 82 selects one tomographic image with the highest contrast from among the plurality of tomographic image data with different set sound velocities. For example, for the individual region A, the selector 82 selects a tomographic image with the highest contrast from among the tomographic images 110, 210, 310 and 410. For example, as shown in FIG. 8B, in a case where the contrast in the tomographic image 310 generated under the condition of the set sound velocity C3 is the highest, the selector 82 selects the tomographic image 310 for the individual region A. The selector 82 outputs the tomographic image data related to the tomographic image 310 to the display controller 9.

When a tomographic image with the highest contrast is selected for one of the individual regions, the controller 13 deletes data other than the data selected by the selector 82 from the image storage 6. For example, in a case where the tomographic image 310 is selected for the individual region A, the controller 13 deletes B-mode ultrasound raster data for generation of tomographic images other than the tomographic image 310, from the image storage 6. In other words, the controller 13 deletes B-mode ultrasound raster data for generation of the tomographic images 110, 210 and 410, from the image storage 6.

Memory resulting from the deletion is used to take in an image of the next individual region.

### (Step S37)

The process from Step 30 to Step 36 is repeated until tomographic images with the highest contrast are selected for all of the individual regions. When a tomographic image with the highest contrast is selected for the individual region A, the controller 13 gives a command to transmit ultrasonic waves to the individual region B, to the transmitter 3. The transmitter 3 transmits ultrasonic waves to the individual region B under the control of the controller 13 (Step S30).

As in the abovementioned process from Step S31 to Step S36, the delaying process is executed on the individual region B at a plurality of set sound velocities, thereby generating a plurality of tomographic image data with different set sound velocities. Then, the contrasts of the plurality of tomographic image data with different set sound velocities are obtained, and tomographic image data with the highest contrast is selected for the individual region B. The controller 13 deletes data other than the selected tomographic image data from the image storage 6. Also on the individual regions C through E, transmission/reception of ultrasonic waves, generation of tomographic image data, calculation of contrast, and selection of tomographic image data are executed on each, and data that has not been selected is deleted from the image storage 6 at each time.

### (Step S38)

When tomographic images with the highest contrast are selected for all of the individual regions (Step S37, Yes), the display controller 9 couples the tomographic image data with the highest contrast in the respective individual regions A through E, thereby reconstructing tomographic image data representing the entire imaging region S.

### (Step S39)

Then, the display controller 9 controls the display 11 to display a tomographic image based on the tomographic image data representing the entire imaging region S.

As described above, by executing transmission/reception of ultrasonic waves, generation of tomographic image data, calculation of contrast and selection of tomographic image data for each individual region and deleting unselected data from the image storage 6 at each time, it is possible to reduce the capacity of the image storage 6. For example, in the case of generating four tomographic image data based on four types of set sound velocities in the entire imaging region S, it is necessary to retain data for four screens (four frames) in the image storage 6. On the contrary, according to the ultrasonic imaging apparatus of Modification 2, it is enough to retain the same number of data as obtained by multiplying the number of the tomographic image data of the individual regions by the number of set sound velocities in the image storage 6, so it is possible to reduce the capacity of the memory necessary for optimization of the sound velocities.

### (Modification 3)

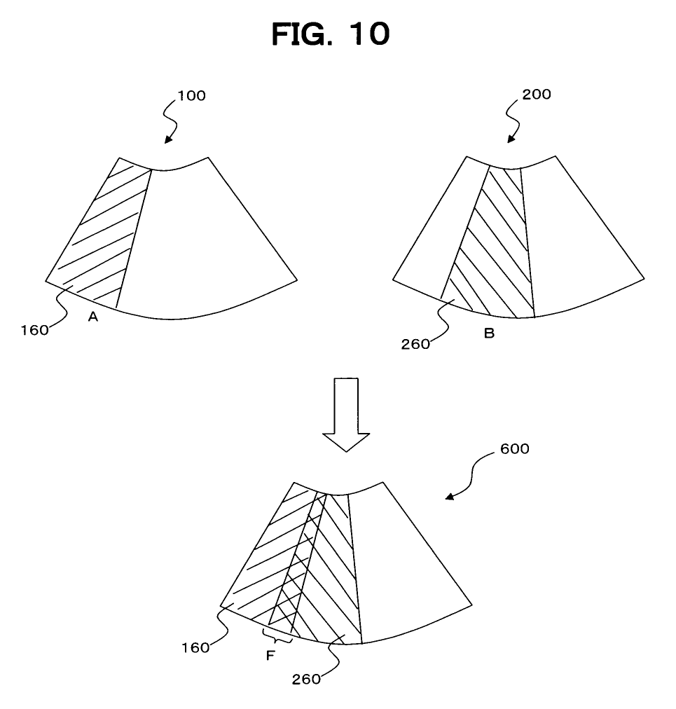

Next, Modification 3 of the ultrasonic imaging apparatus 1 will be described with reference to FIG. 10. FIG. 10 is a view schematically showing tomographic images of individual regions adjacent to each other.

In Modification 3, as in the abovementioned Modification 1 and Modification 2, an entire tomographic image or an entire imaging region is divided into a plurality of individual regions, and a tomographic image with the highest contrast is selected for each of the individual regions. Then, the respective tomographic image data of the individual regions are coupled, whereby tomographic image data indicating the whole is generated. Furthermore, in Modification 3, the entire tomographic image or the whole imaging region is divided so that the individual regions adjacent to each other partially overlap.

In Modification 3, the contrast calculator 81 divides the entire tomographic image into a plurality of individual regions A through E so that the individual regions adjacent to each other partially overlap.

For example, as shown in FIG. 10, the contrast calculator 81 divides the entire tomographic image so that the individual region A and the individual region B partially overlap. This division pattern is previously set in the controller 13. The contrast calculator 81 divides the entire tomographic image in accordance with the division pattern under the control of the controller 13.

In a case where the contrast of a tomographic image 160 included in the tomographic image 100 generated under the condition of the set sound velocity C1 is the highest for the individual region A, and the contrast of a tomographic image 260 included in the tomographic image 200 generated under the condition of the set sound velocity C2 is the highest for the individual region B, the display controller 9 couples the tomographic image 160 and the tomographic image 260. The individual region A and the individual region B partially overlap. The overlapping region is an overlapping region F.

Because the values of the set sound velocities are different between the tomographic image 160 showing the individual region A and the tomographic image 260 showing the individual region B, an image at the connection may be unnatural. Therefore, in Modification 3, the display controller 9 performs blending of the tomographic image data in the individual region A and the tomographic image data in the individual region B, which are included in the overlapping region F, thereby smoothing the connection of the images in the overlapping region F. For example, in the overlapping region in which the individual regions adjacent to each other overlap, the display controller 9 adds the pixel values of each image data in each of the individual regions while changing the ratio of the pixel values of each image data in each of the individual regions depending on locations, thereby generating image data of the overlapping region.

To be specific, the display controller 9 receives the coordinate information of the overlapping region F from the controller 13. Then, for the overlapping region F, while gradually changing the ratio of the pixel values (luminance values) of the tomographic image data in the individual region A and the pixel values (luminance values) of the tomographic image data in the individual region B depending on locations, the display controller 9 adds the pixel values to generate the tomographic image data of the overlapping region F. For example, the display controller 9 makes the ratio of the pixel values of the tomographic image data in the individual region A higher than the ratio of the pixel values of the tomographic image data in the individual region B at locations closer to the individual region A in the overlapping region F, and adds the tomographic image data of the individual region A and the tomographic image data of the individual region B, thereby generating tomographic image data of the overlapping region F. On the other hand, the display controller 9 makes the ratio of the pixel values of the tomographic image data in the individual region B higher than the ratio of the pixel values of the tomographic image data in the individual region A at locations closer to the individual region B, and adds the tomographic image data of the individual region A and the tomographic image data of the individual region B, thereby generating tomographic image data in the overlapping region F.

The display controller 9 couples the respective tomographic image data of the individual regions A through E, and executes the blending process on the overlapping region in which the individual regions overlap, thereby generating tomographic image data indicating the entire image. The display controller 9 then controls the display 11 to display a tomographic image 600 based on the tomographic image data showing the whole.

As described above, by executing the blending process on a part where images with different set sound velocities overlap, it is possible to smooth the connection at the boundary. Thus, even at a boundary where a difference in image quality is large, an image at the boundary is not unnatural, and it is possible to minimize the difference in image quality.

A range for blending and the ratio of the luminance values of the tomographic image data may be arbitrarily changed by the operator using the operation part 12.

What is claimed is:

1. An ultrasonic imaging apparatus, comprising:
a transmitter configured to transmit ultrasonic waves to an object via an ultrasonic probe;
a receiver configured to receive echo signals reflected from the object via the ultrasonic probe, and execute a delaying process on the echo signals in accordance with a plurality of set sound velocities for the delaying process, thereby generating a plurality of reception signals with different set sound velocities;
an image generator configured to generate a plurality of image data with the different set sound velocities, based on the reception signals with the different set sound velocities;
a contrast calculator configured to obtain a contrast of each of the plurality of image data with the different set sound velocities;
a selector configured to select image data with a highest contrast from among the plurality of image data; and
a display controller configured to control a display to display an image based on the image data selected by the selector.

2. The ultrasonic imaging apparatus according to claim 1, wherein:
the contrast calculator divides each of the plurality of image data with the different set sound velocities into a plurality of individual regions, and obtains a contrast of each of the divided individual regions in each of the image data;
the selector selects image data with highest contrasts from among the plurality of image data with the different set sound velocities of the respective individual regions, for the respective individual regions; and
the display controller couples the image data with the highest contrasts selected for the respective individual regions, and controls the display to display an image based on the coupled image data.

3. The ultrasonic imaging apparatus according to claim 1, further comprising:
a controller configured to divide a desired imaging region into a plurality of individual regions, and controls the transmitter to transmit ultrasonic waves to one individual region of the plurality of individual regions; and
a storage, wherein:
the receiver receives echo signals reflected from the one individual region, and executes a delaying process on the echo signals of the one individual region in accordance with the plurality of set sound velocities, thereby generating a plurality of reception signals with the different set sound velocities for the one individual region;
the storage stores the plurality of reception signals with the different set sound velocities for the one individual region;
the image generator generates a plurality of image data with the different set sound velocities for the one individual region, based on the plurality of reception signals with the different set sound velocities;
the contrast calculator obtains a contrast of each of the plurality of image data with the different set sound velocities for the one individual region;
the selector selects image data with a highest contrast from among the plurality of image data of the one individual region;
the controller deletes reception signals relating to image data unselected by the selector, and subsequently executes a series of processes from transmission of ultrasonic waves to the one individual region to deletion of the reception signals, on the respective individual regions, thereby acquiring image data with highest contrasts for the respective individual regions; and
the display controller couples the image data with the highest contrasts acquired for the respective individual regions, and controls the display to display an image based on the coupled image data.

4. The ultrasonic imaging apparatus according to claim 2, wherein:
the display controller adds, in an overlapping region where adjacent individual regions overlap, pixel values of the image data of the respective individual regions while changing a ratio of the pixel values of the image data of the respective individual regions in the overlapping region depending on locations, thereby generating image data of the overlapping region, and controlling the display to display an image based on the coupled image data.

5. The ultrasonic imaging apparatus according to claim 3, wherein:
the display controller adds, in an overlapping region where adjacent individual regions overlap, pixel values of the image data of the respective individual regions while changing a ratio of the pixel values of the image data of the respective individual regions in the overlapping region depending on locations, thereby generating image data of the overlapping region, and controlling the display to display an image based on the coupled image data.

6. The ultrasonic imaging apparatus according to claim 1, further comprising:
a controller configured to change a value of a sound velocity by a specified value with reference to a set sound velocity for generating the image data selected by the selector, thereby newly obtaining a plurality of set sound velocities,
wherein the receiver executes a delaying process on newly received echo signals in accordance with the plurality of set sound velocities having been newly obtained, thereby generating a plurality of reception signals with different set sound velocities.

7. A method for generating an ultrasonic image, comprising:
transmitting ultrasonic waves to an object via an ultrasonic probe;
receiving echo signals reflected from the object via the ultrasonic probe;
executing a delaying process on the echo signals in accordance with a plurality of set sound velocities for the delaying process, thereby generating a plurality of reception signals with different set sound velocities;
generating a plurality of image data with the different set sound velocities, based on the reception signals with the different set sound velocities;
obtaining a contrast of each of the plurality of image data with the different set sound velocities;
selecting image data with a highest contrast from among the plurality of image data; and
displaying an image based on the selected image data.

8. The method for generating an ultrasonic image according to claim 7, wherein:
each of the plurality of image data with the different set sound velocities is divided into a plurality of individual regions, and a contrast of each of the divided individual regions is obtained in each of the image data;
image data with highest contrasts among the plurality of image data with the different set sound velocities of the respective individual regions are selected for the respective individual regions; and
the image data with the highest contrasts selected for the respective individual regions are coupled, and an image based on the coupled image data is displayed.

9. The method for generating an ultrasonic image according to claim 7, wherein:
a desired imaging region is divided into a plurality of individual regions, and ultrasonic waves are transmitted to one individual region of the plurality of individual regions;
echo signals reflected from the one individual region are received, and a delaying process is executed on the echo signals of the one individual region in accordance with the plurality of set sound velocities, whereby a plurality of reception signals with the different set sound velocities are generated for the one individual region;
the plurality of reception signals with the different set sound velocities for the one individual region are stored into a storage;
a plurality of image data with the different set sound velocities are generated for the one individual region based on the plurality of reception signals with the different set sound velocities;
a contrast of each of the plurality of image data with the different set sound velocities is obtained for the one individual region;
image data with a highest contrast is selected from among the plurality of image data of the one individual region;
reception signals relating to image data unselected by the selector among the plurality of stored reception signals are deleted;
a series of processes from transmission of ultrasonic waves to the one individual region to deletion of the reception signals are executed on the respective individual regions, whereby image data with highest contrasts are acquired for the respective individual regions; and
the image data with the highest contrasts acquired for the respective individual regions are coupled, and an image based on the coupled image data is displayed.

10. The method for generating an ultrasonic image according to claim 8, wherein:
in an overlapping region where adjacent individual regions overlap, pixel values of the image data of the respective individual regions are added while a ratio of the pixel values of the image data of the respective individual regions in the overlapping region is changed depending on locations, whereby image data of the overlapping region is generated, and an image based on the coupled image data is displayed.

11. The method for generating an ultrasonic image according to claim 9, wherein:
in an overlapping region where adjacent individual regions overlap, pixel values of the image data of the respective individual regions are added while a ratio of the pixel values of the image data of the respective individual regions in the overlapping region is changed depending on locations, whereby image data of the overlapping region is generated, and an image based on the coupled image data is displayed.

12. The method for generating an ultrasonic image according to claim 7, wherein:
a value of a sound velocity is changed by a specified value with reference to a set sound velocity for generating the image data selected by the selector, whereby a plurality of set sound velocities are newly obtained;
a delaying process is executed on newly received echo signals in accordance with the plurality of set sound velocities having been newly obtained, whereby a plurality of reception signals with different set sound velocities are generated; and
every time the image data is selected, a plurality of set sound velocities with a value of a sound velocity changed by a specified value are obtained with reference to the set sound velocity for generating the selected image data.
